(19) United States
(12) Patent Application Publication
(10) Pub. No.: US 2011/0028808 A1
(43) Pub. Date: Feb. 3, 2011

(54) METHOD AND APPARATUS FOR EXAMINATION OF CANCER, SYSTEMIC LUPUS ERYTHEMATOSUS (SLE), OR ANTIPHOSPHOLIPID ANTIBODY SYNDROME USING NEAR-INFRARED LIGHT

(75) Inventors: Hirohiko Kuratsune, Osaka (JP); Akikazu Sakudo, Osaka (JP); Junzo Nojima, Osaka (JP); Kazuyoshi Ikuta, Osaka (JP); Yasuyoshi Watanabe, Osaka (JP); Yukiko Kato, Osaka (JP); Takanori Kobayashi, Osaka (JP); Seiki Tajima, Osaka (JP)

Correspondence Address: JHK LAW, P.O. BOX 1078, LA CANADA, CA, 91012-1078, US

(73) Assignees: Hirohiko Kuratsune, Osaka (JP); Fatigue Science Laboratory Inc., Osaka (JP); Osaka University, Osaka (JP)

(21) Appl. No.: 12/307,263

(22) Filed: Dec. 31, 2008

(30) Foreign Application Priority Data
Jul. 6, 2006 (JP) JP-2006-186223

(51) Int. Cl. A61B 6/00; G01J 5/02

(52) U.S. Cl. 600/322; 250/339.07

(57) ABSTRACT

An object of the present invention is to provide apparatuses for examining/diagnosing clinical disease of cancer, systemic lupus erythematosus (SLE), or antiphospholipid antibody syndrome. Blood, blood-derived component, urine, sweat, nail, skin, or hair is irradiated with light having a wavelength of 400 to 2,500 nm or a part of the range, of which the reflection light, the transmission light, or the transmission reflection light is then detected to give spectroscopic absorbance data, and afterward a previously prepared analysis model is used to analyze the absorbance over the whole wavelengths or at a specific wavelength for the measurement to allow the achievement of the object.

### TECHNICAL FIELD

The present invention relates to a method for clinical blood examination and identification using near-infrared light; and the apparatus used for the method, particularly to the method for clinical examination of cancer, systemic lupus erythematosus (SLE), or antiphospholipid antibody syndrome; and the apparatus used for the method.

Additionally, the present invention claims priority from Japanese Patent Application Number 2006-186223, the content of which is incorporated herein by reference.

### BACKGROUND ART

Recently, the preliminary testing of cancer is carried out by using as an index the level of tumor markers [CA19-9 (carbohydrate antigen-19-9), CEA (cancer embryonic antigen), AFP (α-fetoprotein), PIVKA-II, PSA (prostatic specific antigen), CA125 (carbohydrate antigen 125)] in blood. If the preliminary testing is positive, the definite diagnosis and malignancy of cancer are examined using microscopy of tissue biopsy. However, as there are no tumor markers specific to cancer, false-positive rate is high. Thus, the improved method for cancer clinical testing is greatly beneficial to a synthetic judgment of cancer.

Antiphospholipid antibody (PL) includes anticardiolipin antibody (CL), lupus anticoagulant activity (LAC), and false-positive Wassermann reaction and the like, and antiphospholipid antibody syndrome is called the cases where thrombosis of artery/vein, thrombopenia, habitual abortion/stillbirth/intrauterine fetal death, and the like are clinically developed while having these antibodies.

Antiphospholipid antibody is often confirmed in collagen disease and autoimmune disease including systemic lupus erythematosus (SLE) (secondary), but also exists in primary antiphospholipid antibody syndrome. Antiphospholipid antibody syndrome is identified by using clinical picture and immunological testing (Non-Patent Document 1).

Their diagnostic criteria is based on that the clinical picture shows venous thrombus, arterial thrombus, iterative abortion or fetal death, and platelet depletion, and the sample corresponds to at least any of IgG-type CL-antibody (20 GPL or more), LA-positive, and IgM-type CL-antibody positive+LA-positive through immunological examination.

Thus, the improved method for clinical testing relating to antiphospholipid antibody syndrome is greatly beneficial to a synthetic judgment of antiphospholipid antibody syndrome.

In the meantime, in recent years, componential analyses have been performed in various fields using near-infrared light. For example, a host is irradiated with visible light and/or near-infrared light to detect a wavelength band absorbed by a specific component, thereby to analyze quantitatively various specific components. Concretely, the sample is put in a quartz cell, and then irradiated with visible light and/or near-infrared light having a wavelength of 400 to 2500 nm using the near-infrared spectroscope (such as the near-infrared spectroscope NIRSystem6500 made by NIRECO corp.) to assay the reflection light, the transmission light, or the a transmission reflection light. Generally speaking, near-infrared light, which is a low energy of electromagnetic wave to have so small an absorption coefficient that it is hardly scattered by a substance, gives no damage to a sample to allow collecting intact chemical/physical information about the sample. Concretely, the light such as the transmission light from the irradiated sample can be detected to collect the absorbance data about the sample, which is then analyzed multivariately to collect promptly information about the sample, for example, to grasp the change of a biomolecule in structure and function directly and in real time. The conventional technique for such near-infrared spectrometry is described, for example, in Patent Document No. 1 and No. 2 below. Patent Document No. 1 discloses a method for using visible and near-infrared light to collect the information from a subject, concretely, a method to identify a group to which an unknown subject belongs, a method to identify the unknown subject, and a method to monitor the aging change of the subject in real time. Patent Document No. 2 discloses a method for the diagnosis of bovine mastitis by the measurement of somatic cells in milk or bovine dugs after the absorbance data obtained is analyzed multivariately using absorption band for water molecule in visible light and/or near-infrared light range.
Patent Document No. 1: Japanese Patent Application Laid-open No. 2002-5827
Patent Document No. 2: International Laid-open Patent Publication WO01/75420
Patent Document No. 3: Japanese Unexamined Patent Publication No. 2003-500648
Non-Patent Documents 1: Harris, E. N. Antiphospholipid antibodies. Br J Haematol, 74:1, 1990.

### DISCLOSURE OF THE INVENTION

### Problem to be Solved by the Invention

An object of the present invention is to provide a method for clinical examination of cancer, systemic lupus erythematosus (SLE), or antiphospholipid antibody syndrome by irradiating blood, blood-derived component, urine, sweat, nail, skin, or hair with near-infrared light and its apparatus.

### Means for Solving the Problem

The present inventors have keenly continued examinations to attain the problem described above and have completed the following present inventions.

1. A method for identification of a clinical disease selected from following items, comprising the steps of: irradiating collected blood, blood-derived component, urine, sweat, nail, skin, or hair with light having a wavelength of 400 to 2500 nm or apart of the range, of which a reflection light, a transmission light, or a transmission reflection light is then detected to give spectroscopic absorbance data, and
analyzing an absorbance over the whole wavelengths or at a specific wavelength for a measurement by using a previously prepared analysis model.
1) Cancer
2) Systemic lupus erythematosus (SLE)
3) Antiphospholipid antibody syndrome

2. The method for identification according to Item 1, comprising the steps of:
irradiating the blood, blood-derived component, urine, sweat, nail, skin, or hair collected from normal persons and patients with clinical disease light having the wavelength of 400 to 2500 nm or apart of the range, of which the reflection light, the transmission light, or the transmission reflection light is then detected to give spectroscopic absorbance data, and
analyzing the difference wavelength after assaying the difference in absorbance between normal persons and patients with clinical disease.

3. The method for identification according to Item 2, wherein an analysis method of the difference wavelength adopts a principal component analysis or a SIMCA method.

4. The method for identification according to any one of Items 1 to 3, wherein a perturbation is given to the collected blood, blood-derived component, urine, sweat, nail, skin, or hair.

5. The method for identification according to any one of the Items 1 to 4, wherein the absorption spectrum detected is the transmission light.

6. The method for identification according to any one of the Items 1 to 5, wherein the absorption spectroscopic data at two or more wavelengths, which are selected from a plurality of ±5 nm wavelength ranges of each wavelength selected from the group consisting of 625-675 nm, 775-840 nm, 910-950 nm, 970-1010 nm, 1020-1060 nm, and 1070-1090 nm, are used for the identification of a clinical disease of cancer.

7. The method for identification according to any one of the Items 1 to 5, wherein the absorption spectroscopic data at two or more wavelengths, which are selected from a plurality of ±5 nm wavelength ranges of each wavelength selected from the group consisting of 740-780 nm, 790-840 nm, 845-870 nm, 950-970 nm, 975-1000 nm, 1010-1050 nm, and 1060-1100 nm, are used for the identification of the clinical disease of systemic lupus erythematosus (SLE).

8. The method for identification according to any one of the Items 1 to 5, wherein the absorption spectroscopic data at two or more wavelengths, which are selected from a plurality of ±5 nm wavelength ranges of each wavelength selected from the group consisting of 600-650 nm, 660-690 nm, 780-820 nm, 850-880 nm, 900-920 nm, 925-970 nm, and 1000-1050 nm, are used for the identification of the clinical disease of antiphospholipid antibody syndrome.

9. A method for diagnosis of the clinical disease selected from following items, wherein a finger or an ear of a patient with clinical disease is irradiated with light having a wavelength of 400 to 2500 nm or apart of the range, of which the reflection light, the transmission light, or the transmission reflection light is then detected to give spectroscopic absorbance data, and afterward the previously prepared analysis model is used to analyze the absorbance over the whole wavelengths or at a specific wavelength for the measurement.
1) Cancer
2) Systemic lupus erythematosus (SLE)
3) Antiphospholipid antibody syndrome

10. The method for diagnosis according to Item 9, wherein the finger or ear of a normal person and a patient with clinical disease is irradiated with light having the wavelength of 400 to 2500 nm or apart of the range, of which the reflection light, the transmission light, or the transmission reflection light is then detected to give spectroscopic absorbance data, and afterward the analysis model assays the difference of the absorbance between the normal person and the patient with clinical disease to analyze the difference wavelength.

11. An examination/diagnosis apparatus for a clinical disease selected from following items, comprising:
an irradiating means for irradiating blood, blood-derived component, urine, sweat, nail, skin, or hair with the light having a wavelength of 400 to 2500 nm or apart of the range; a spectroscoping means for spectroscoping before or after irradiation and a detecting means for detecting the reflection light, the transmission light, or the transmission reflection light of the light irradiated on the blood, blood-derived component, urine, sweat, nail, skin, or hair; and
a data analyzing means for using a previously formed analysis model to analyze the absorbance(s) at the whole or specific wavelength used for the measurement in the absorbance spectroscopic data obtained by the detection, thereby to analyze qualitatively and quantitatively about the blood, blood-derived component, urine, sweat, nail, skin, or hair.
1) Cancer
2) Systemic lupus erythematosus (SLE)
3) Antiphospholipid antibody syndrome

12. The apparatus according to claim 11, wherein the blood, blood-derived component, urine, sweat, nail, skin, or hair of normal persons and patients with clinical disease is irradiated the with lights having the wavelength of 400 to 2500 nm or a part of the range, of which the reflection light, the transmission light, or the transmission reflection light is then detected to give spectroscopic absorbance data, and afterward the analysis model is prepared by assaying the difference of the absorbance between the normal person and the patient with clinical disease to analyze the difference wavelength.

13. The apparatus according to Item 12, wherein the analysis method of the difference wavelength adopts the principal component analysis or the SIMCA method.

14. The apparatus according to any one of the Items 11 to 13, wherein the absorption spectrum detected is the transmission light.

15. The apparatus according to any one of the Items 11 to 14, wherein the absorption spectroscopic data at two or more wavelengths, which are selected from a plurality of ±5 nm wavelength ranges of each wavelength selected from the group consisting of 625-675 nm, 775-840 nm, 910-950 nm, 970-1010 nm, 1020-1060 nm, and 1070-1090 nm, are used for the clinical disease of cancer.

16. The apparatus according to any one of the Items 11 to 14, wherein the absorption spectroscopic data at two or more wavelengths, which are selected from a plurality of ±5 nm wavelength ranges of each wavelength selected from the group consisting of 740-780 nm, 790-840 nm, 845-870 nm, 950-970 nm, 975-1000 nm, 1010-1050 nm, and 1060-1100 nm, are used for the clinical disease of systemic lupus erythematosus (SLE).

17. The apparatus according to any one of the Items 11 to 14, wherein the absorption spectroscopic data at two or more wavelengths, which are selected from a plurality of ±5 nm wavelength ranges of each wavelength selected from the group consisting of 600-650 nm, 660-690 nm, 780-820 nm, 850-880 nm, 900-920 nm, 925-970 nm, and 1000-1050 nm, are used for the clinical disease of antiphospholipid antibody syndrome.

### Effect of the Invention

The present invention can examine/identify simply, promptly, and highly accurately the clinical examination of cancer, systemic lupus erythematosus (SLE), or antiphospholipid antibody syndrome and can widely be used for identification of a clinical examination. In particular, the present invention is useful when there is need to examine a large number of test samples or objects all at once, and the like due to allowing its simple and prompt examination. In addition, as the examination can be noninvasively performed to an object, prompt and simple clinical examination can be carried out with no pain given to an object.

### BRIEF DESCRIPTION OF THE DRAWINGS

FIG. 1-1 shows a measurement apparatus for an absorbing spectrum.

FIG. 1-2 shows a result of using the analysis model for a principal component analysis (PCA) with a near-infrared spectrum in Test sample (hepatic cancer patient; 76, normal person; 31).

FIG. 1-3 shows a result identifying the masked samples (hepatic cancer patient; 21, normal person; 20) using a principal component analysis (PCA) with the near-infrared spectrum.

FIG. 1-4 shows a loading of the analysis model for a principal component analysis with the near-infrared spectrum in Test sample (hepatic cancer patient; 76, normal person; 31).

FIG. 1-5 shows a condition for PCA.

FIG. 2-1 shows a result of using a SIMCA model in Test sample (hepatic cancer patient; 76, normal person; 31) with a near-infrared spectrum.

FIG. 2-2 shows a result of using the SIMCA model in masked sample (hepatic cancer patient; 21, normal person; 20) by the near-infrared spectrum.

FIG. 2-3 shows a prediction result of cancer through the SIMCA model.

FIG. 2-4 shows a discriminating power of the SIMCA model in masked sample (hepatic cancer patient; 76, normal person; 31) by the near-infrared spectrum.

FIG. 2-5 shows a condition for the SIMCA.

FIG. 3-1 shows a result of using the analysis model for a principal component analysis (PCA) in Test sample (SLE; 97, normal person; 41) by the near-infrared spectrum.

FIG. 3-2 shows a result identifying the masked samples (SLE; 25, normal person; 10) using a principal component analysis (PCA) with the near-infrared spectrum.

FIG. 3-3 shows a loading of the analysis model for a principal component analysis in Test sample (SLE; 97, normal person; 41) by the near-infrared spectrum.

FIG. 3-4 shows a condition for PCA.

FIG. 4-1 shows a result of using the SIMCA model in Test sample (patient with SLE; 97, normal person; 41) by the near-infrared spectrum.

FIG. 4-2 shows a result of using the SIMCA model in masked sample (patient with SLE; 25, normal person; 10) by the near-infrared spectrum.

FIG. 4-3 shows the prediction result of SLE through the SIMCA model.

FIG. 4-4 shows the discriminating power of the SIMCA model in Test sample (patient with SLE; 97, normal person; 41) with the near-infrared spectrum.

FIG. 4-5 shows a condition for the SIMCA.

FIG. 5-1 shows a result of using the analysis model for a principal component analysis (PCA) in Test sample (APLs(+); 51, APLs(−); 41) by the near-infrared spectrum.

FIG. 5-2 shows a result identifying the masked samples (APLs(+); 15, APLs(−); 15) using a principal component analysis (PCA) by the near-infrared spectrum.

FIG. 5-3 shows a loading result of the analysis model for a principal component analysis in Test sample (APLs(+); 51, APLs(−); 41) by the near-infrared spectrum.

FIG. 5-4 shows a condition for PCA.

FIG. 6-1 shows a result of using the SIMCA model in Test sample (APLs positive patient; 51, APLs negative patient; 41) by the near-infrared spectrum.

FIG. 6-2 shows a result of using the SIMCA model in masked sample (APLs positive patient; 15, APLs negative patient; 15) by the near-infrared spectrum.

FIG. 6-3 shows a discriminating power of the SIMCA model in Test sample (APLs positive patient; 51, APLs negative patient; 41) by the near-infrared spectrum.

FIG. 6-4 shows a prediction result of the APLs positive patients through the SIMCA model.

FIG. 6-5 shows a condition for SIMCA.

### DESCRIPTION OF THE PREFERRED EMBODIMENT

One aspect of the present invention is a method that collects the information of clinical disease, particularly the diagnostic result regarding cancer, systemic lupus erythematosus (SLE), or antiphospholipid antibody syndrome from blood, blood-derived component, urine, sweat, nail, skin, or hair, wherein the blood, blood-derived component, urine, sweat, nail, skin, or hair are irradiated with light having a wavelength of 400 nm to 2500 nm, a near-infrared light, or a part of the range, of which the reflection light, the transmission light, or the transmission reflection light is then detected to give spectroscopic absorbance data, and afterward the previously prepared analysis model is used to analyze the absorbance at the whole or specific wavelength used for the measurement.

In the present invention, blood or blood-derived component may be blood collected for examination, one fractionated from the blood, blood serum or blood plasma. Blood or blood-derived component is stored in a glass test tube or plastic test tube, and the stored tube is subjected to the measurement. Further, the present invention includes the case where the direct measurement of human's blood is non-invasively carried out. The expression reading “non-invasively carried out” means that a finger, ear or the like is irradiated with near-infrared light without collecting blood to give the spectroscopic absorbance data and to carry out the identification of these data.

Additionally, urine, sweat, nail, skin, or hair and the extract from them are obtained by the known method per se.

In the present invention, the information of clinical diseases, particularly diagnostic result, obtained by irradiating blood, blood-derived component, urine, sweat, nail, skin, or hair, particularly blood or blood-derived component with near-infrared light, is intended especially for cancer, systemic lupus erythematosus (SLE), or antiphospholipid antibody syndrome. In the examples of the present invention, hepatic cancer is shown as an exemplification of the cancer, but if the method of the present invention is widely used, other cancers except this exemplified cancer can also be applied.

Such cancer includes lung cancer (squamous cell cancer of the lung, adenocarcinoma of the lung, small cell lung cancer), thymoma, thyroid cancer, prostate cancer, kidney cancer, bladder cancer, colon cancer, rectum cancer, esophagus cancer, cecal cancer, ureter cancer, breast cancer, uterine cervix cancer, brain cancer, tongue cancer, pharynx cancer, nasal cavity cancer, larynx cancer, stomach cancer, bile duct cancer, testicle cancer, ovary cancer, endometrial cancer, metastatic bone cancer, malignant melanoma, bone cancer, malignant lymphoma, plasmacytoma, liposarcoma, and the like.

Further, in the examples of the present invention, antiphospholipid antibody syndrome is exemplified, and this syndrome clinically develops thrombosis of artery/vein, thrombopenia, habitual abortion/stillbirth/intrauterine fetal death, and the like, while having the antibody of antiphospholipid antibody (PL), such as anticardiolipin antibody (CL), lupus anticoagulant factor (LAC), and false-positive Wassermann reaction. Antiphospholipid antibody syndrome often is confirmed in collagen disease, autoimmune disease, including systemic lupus erythematosus (SLE) (secondary), but also there is primary antiphospholipid antibody syndrome.

In the present invention, blood, blood-derived component, urine, sweat, nail, skin, or hair, particularly blood or blood-derived component, is irradiated with near-infrared light to compare a normal person with each patient with clinical diseases (cancer, systemic lupus erythematosus (SLE), or antiphospholipid antibody syndrome). Thus, in the present invention, the comprehensive abnormality obtained from the comparison can be identified, allowing to be applied to the identification of clinical diseases.

It is preferable to establish an analysis model for the identification in the present invention. The present invention can obtain the information of clinical diseases, particularly the result of identification/diagnosis for clinical diseases by comparison with this analysis model. The analysis model can be prepared with the following method. The blood or blood-derived component of a normal person and a patient with clinical disease (cancer, systemic lupus erythematosus (SLE), or antiphospholipid antibody syndrome) are irradiated with light having a wavelength of 400 nm to 2500 nm or a part of the range, of which the reflection light, the transmission light, or the transmission reflection light is then detected to give spectroscopic absorbance data. A difference of the absorbance between a normal person and a patient with clinical disease (cancer, systemic lupus erythematosus (SLE), or antiphospholipid antibody syndrome) is analyzed to give the analysis model by the statistical analysis of the difference wavelength. Further, in antiphospholipid antibody syndrome, the difference of the absorbance in a positive and a negative antiphospholipid antibody is also analyzed to prepare the analysis model by the statistical analysis of the difference wavelength.

An examination/diagnosis apparatus for obtaining the information of the clinical diseases of the present invention, comprising: an irradiation means for irradiating the test sample with light having a wavelength of 400 nm to 2500 nm or apart of the range; a spectroscopic means for spectroscoping before or after irradiation and a detection means for detecting the reflection light, the transmission light, or the transmission reflection light of the light irradiated on the said test samples; a data analyzing means for using a previously formed analysis model to analyze the absorbance(s) at the whole wavelength or the specific wavelength measured in the spectroscopic absorbance data obtained by the detector, thereby to examine qualitatively and quantitatively a biochemical substance of the test samples.

Outline of Spectrometry

The examination/diagnosis/identification of cancer, systemic lupus erythematosus (SLE), or antiphospholipid antibody syndrome of the test sample is carried out through the procedure wherein (a) blood, blood-derived component, urine, sweat, nail, skin, or hair as a test sample, particularly blood or blood-derived component collected, is irradiated with light having a wavelength of 400 nm to 2500 nm or a part of the range, (b)its reflection light, its transmission light, or its transmission reflection light is then detected to give spectroscopic absorbance data, and afterward (c) a previously prepared analysis model is used to analyze the absorbance over the whole wavelengths or at a specific wavelength for the measurement.

The present invention is primarily characterized by allowing simply, promptly, and highly accurately obtaining the information, particularly the diagnostic result, of cancer, systemic lupus erythematosus (SLE), or antiphospholipid antibody syndrome of the test sample, and the cancer or antiphospholipid antibody syndrome can also be assayed noninvasively to a living body. The range of wavelength, within which the test sample is irradiated, is from 400 nm to 2500 nm or a part of the range (for example, 600 to 1100 nm). This range of wavelength may be set as one or a plurality of fractional ranges of wavelength which contain lights having wavelengths required for the examination/diagnosis/identification through the analysis model prepared.

The light source to be used can include, but is not limited to, a halogen lamp, a LED and the like. Light emitted from the light source is irradiated on a test sample directly or through an irradiating means such as a fiber probe. A pre-spectroscopic system spectroscoping lights through a spectroscope may be employed before irradiating the test sample, or a post-spectroscopic system spectroscoping lights after irradiating the test sample may be employed. The pre-spectroscopic system is carried out by one method of using a prism to spectroscope lights from a light source all at once, or by another method of changing the slit width of the diffraction grating to change wavelengths consecutively. The latter method resolves lights from a light source into certain wavelength widths to irradiate a test sample with continuous wavelength light which is continuously varied in wavelength. For example, it is possible that the light within the range of 600-1000 nm is resolved by 1 nm of wavelength resolution, and the test sample is irradiated with light consecutively varied in wavelength by every resolution of 1 nm.

The reflection light, the transmission light, or the transmission reflection light of the light irradiated on the test sample is detected by a detector to provide an intact spectroscopic absorbance data. The intact spectroscopic absorbance data may directly be used to examine/diagnose/identify through an analysis model. The data is preferably treated to convert, for example, by using a spectroscopic procedure or a multivariate procedure to resolve peaks in the obtained spectrum into the elemental peaks, and the converted spectroscopic absorbance data is then used for the examination/diagnosis/identification through the analysis model.

The spectroscopic procedure includes secondary differentiation or Fourier transform, and the multivariate procedure exemplifies Weblet transform or neural network method, but they are not particularly limited.

Additionally, in the spectrometry by the present apparatus, a perturbation can be given to the test sample, which is provided by adding the certain conditions to the test sample.

Data analysis method (preparation of analysis model) The apparatus of the present invention adopts an analysis model to analyze the absorbance at a particular wavelength (or over whole measure wavelengths) in the spectroscopic absorbance data obtained, thereby to assay the degree of abnormality of cancer, systemic lupus erythematosus (SLE), or antiphospholipid antibody syndrome in the test sample. Thus, it is preferable that the analysis model is previously prepared in order to finally apply to the clinical examination of cancer, systemic lupus erythematosus (SLE), or antiphospholipid antibody syndrome. It is needless to say that the analysis model may also be simultaneously prepared with the spectroscopic measurement.

Preferably, the analysis model is previously prepared before the measurement. Alternatively, the spectroscopic data obtained by the measurement may be divided into one data for the preparation of the analysis model and another data for assay, and the analysis model obtained on the basis of the data for the preparation of the analysis model may be used to assay. For example, when a large number of test samples are examined all at once, a part of them is used to prepare the analysis model. In other words, the analysis model is simultaneously prepared with the spectroscopic measurement. The procedure can prepare the analysis model without teacher's data, allowing coping with both the quantitative model and the qualitative model.

The analysis model can be prepared by multivariate analysis. For example, cancer, systemic lupus erythematosus (SLE), or antiphospholipid antibody syndrome is anticipated for analysis of blood by singular-value decomposition of a data matrix storing the absorption spectrum over the whole wavelengths obtained by the spectroscopic measurement into scores and loadings to extract principal components estimating the fluctuation of cancer, systemic lupus erythematosus (SLE), or antiphospholipid antibody syndrome from the blood (principal component analysis). The principal component is represented, in descending order of dispersion (that is, dispersion of data groups), as principal component 1, principal component 2, principal component 3, . . . . This allows qualitatively analyzing the fluctuation of cancer, systemic lupus erythematosus (SLE), or antiphospholipid antibody syndrome. Additionally, this allows multiple regression analysis to use independent components which are low in collinearity (=high correlation among explanatory variables). The multiple regression analysis can be applied by allocating score or loading to the explanatory variables, and the amount of the substance related to cancer, systemic lupus erythematosus (SLE), or antiphospholipid antibody syndrome to the objective variables. This can prepare an analysis model which bases on the absorption spectrum over the whole or specific wavelength used for the measurement to estimate the amount of the substance related to cancer, systemic lupus erythematosus (SLE), or antiphospholipid antibody syndrome.

These serial procedures are established as Principal Component Regression (PCR) or PLS (Partial Least Squares) regression (see: Yukihiro Ozaki, Akihumi Uda, Toshio Akai, “Multivariate Analysis for Chemist-Introduction to Chemometrix”, Kodansha Co., Ltd., 2002).

Regression analysis includes additionally CLS (Classical Least Squares) and Cross Variation. In antiphospholipid antibody syndrome, the analysis model can be similarly prepared for between antiphospholipid antibodies positive and negative.

The analysis model using the multivariate analysis can be prepared by employing a self-made software or a commercially available multivariate analysis software. Further, a software specialized in the intended use may be prepared to allow prompt analysis.

An analysis model prepared by using such multivariate analysis software is stored as a file and then called when a test sample containing blood or blood-derived component is assayed, which allows quantitative or qualitative assay of the test sample, using the analysis model. Thus, this allows simple and prompt clinical examination of the test sample of cancer, systemic lupus erythematosus (SLE), or antiphospholipid antibody syndrome. A plurality of analysis models including a quantitative model and a qualitative model are stored as files, which are preferably updated into appropriate ones.

Thus, the examination/diagnosis/identification program (analysis software) of the present invention allows a computer to execute preparation or updating of the analysis model, or examination/diagnosis/identification of each clinical disease, based on spectroscopic data of a sample through the use of the prepared analysis model. The program of the present invention can be provided as a recording medium in which program is readable by the computer.

The analysis model prepared can determine what wavelength of light is necessary for assay using the analysis model. The present apparatus can be simplified in construction by designing to irradiate a test sample with a single or a plurality of wavelength ranges of lights so determined.

Suitable Test Sample Measurement Method and Data Analysis by the Present Invention

In the spectroscopic measurement by the present invention, a perturbation can be given to the test sample, which is provided by adding the certain conditions to the test sample. Further, in the data analysis by the present apparatus, the data analysis which elicits the effect of this perturbation is suitably exemplified.

Perturbation

The term “perturbation” means that, regarding a certain condition, plural kinds/conditions are set for measurement, which induces the change in absorbance of test samples, thereby obtaining a plurality of different spectroscopic data from each other. Examples of the conditions include a change in concentration (include, dilution in concentration), repeated irradiation with lights, extension of irradiation time, addition of electromagnetic force, change of light path length, temperature, pH, pressure, mechanical vibration, any of other conditions which is modified to induce physical and chemical change, or a combination with them, and are roughly divided into (1) conditions relating to the ways of irradiating lights, and (2) conditions relating to the ways of the arrangement/preparation of test samples. The examples of (1) and (2) are represented as repeated irradiation with lights and dilution in concentration, respectively, and are explained as follows.

The repeated irradiation with lights is the method of repeatedly irradiating the test sample with lights continuously or at certain time intervals to give the perturbation of plural measurements, thereby to perform spectral measurements of the test sample. For example, by irradiating the test sample with lights three times continuously, pluralities of spectroscopic data which are different from each other are obtained with the subtle change in absorbance (fluctuation) of the test sample. These spectroscopic data can be used for a multivariate analysis such as a principal component analysis, a SIMCA method or a PLS to improve an analytical accuracy, thus allowing high accurate detection/diagnosis. In addition, when normal spectrum is measured, measurements are carried out by irradiating the test sample with lights more than once, but this aim is to get an average value, which is different from the above-mentioned “perturbation”.

It is considered that the absorbance change of the test sample by a perturbation is caused by the change (fluctuation) in the absorption of water molecule in the test sample. Namely, It is considered that three times-repeated light irradiation as a perturbation causes subtly different kinds of changes in the response and the absorption of the water on each of the first, the second and third attempts and as a result, the fluctuation is created in spectrum.

A principal component analysis or a SIMCA method based on each absorbance spectroscopic data obtained by such three times-repeated irradiation is used to allow favorable and qualitative analyzing derivation from a patient with cancer, systemic lupus erythematosus (SLE), or antiphospholipid antibody syndrome of each test sample in the examples.

Further, in the case of the three times-repeated irradiation, at least two data out of the three spectroscopic absorbance data obtained are used to perform a principal component analysis, which allows favorably classifying each test sample. Thus, examination/diagnosis/identification can be carried out highly accurately. Though the number of irradiations is not especially limited to three times, the number of irradiations is preferably about three times from the viewpoint of a complexity of data analysis.

On the other hand, in the perturbation by the dilution in concentration, the test samples diluted in a few steps are prepared, and a spectrometry of each sample is performed. This allows obtaining a plural spectroscopic data from a test sample, and these spectroscopic data allow highly accurate examination/diagnosis using the multivariate analysis. As an example for the multivariate analysis of this case, PLS regression analysis, in which a dilution ratio per each test sample is identified as an objective variable, is performed and regression vectors obtained are then classified using pattern recognition such as a SIMCA method. A class-identifying model so prepared is used to identify/classify to which class of regression vectors (pattern) the regression vectors of the test sample is close, which allows examination/diagnosis.

The number of dilutions and the degree of the dilution are not especially limited. The fluctuation in the spectrum obtained may be created by the perturbation caused by the dilution of concentration, which allows these numerical values to be set optionally.

As with the perturbation conditions except the dilution of concentration and the repeated irradiations, plural kinds/conditions can be set for each condition to measure spectrum so that the fluctuation can be created in the spectrum obtained (see, Japanese Patent Application No. 2003-379517).

Data Analysis Method Eliciting Perturbation Effect

The term “data analysis method eliciting perturbation effect” is that a plurality of spectroscopic data obtained by the perturbation per test sample are used to prepare the analysis models and the analysis models are used to perform the data analysis. The illustrative examples of the data analysis methods include three methods described below.

(a) Quantitative analysis: A method for quantitating the amount of objective substances, such as the amount of particular biochemical substances, in a test sample through the use of a quantitative model prepared by regression analysis such as PLS method. The quantitative model is prepared through the use of a plurality of spectroscopic data obtained by the perturbation per test sample.

(b) Qualitative analysis 1: a method for assaying a test sample through the use of a qualitative model prepared by a class-identifying analysis such as a principal component analysis and a SIMCA method. The qualitative model is prepared through the use of a plurality of spectroscopic data obtained by the perturbation per test sample.

(c) Qualitative analysis 2: a method for assaying a test sample through the use of a qualitative model prepared, wherein (1) a regression analysis, in which each value of the perturbation (each value modified in the condition to give the perturbation) such as a dilution value (a dilution ratio) is identified as an objective variable, is performed, (2) a class-identifying analysis of the regression vectors obtained by that analysis, such as a principal component analysis and a SIMCA method is performed. The regression analysis is performed using a plurality spectroscopic data obtained by the perturbation per test sample, as described above.

Specific Construction of the Measurement Apparatus of the Present Invention

The examination/diagnosis system of the apparatus of the present invention comprises four elements: a probe (irradiating part); a spectroscoping/detecting part; a data analyzing part; and a result displaying part.

Probe (Irradiating Part)

The probe has a function of introducing light (having a wavelength of 400 nm-2500 nm or a part thereof) from a light source such as a halogen lamp or LED into a test sample intended for measurements. There is mentioned as the fiber probe a system for irradiating an object (test sample) with lights via a flexible optical fiber. Generally, the probe for the near-infrared spectroscope can be produced inexpensively and is available at low cost.

In addition, the system may be designed to irradiate directly an object (test sample) with lights emitted from a light source. In that case, probe is not needed and the light source serves as a light irradiating means.

As described above, the analysis model prepared can determine what wavelength of light is necessary for the examination/diagnosis/identification of cancer, systemic lupus erythematosus (SLE), or antiphospholipid antibody syndrome through the use of the analysis model. The present apparatus can be simplified in construction by being designed to irradiate a test sample with a single or a plurality of wavelength ranges of lights, thus determined.

Further, in a preferred embodiment, the present apparatus carries out a spectroscopic measurement while giving a perturbation and is preferable to be equipped with a structure required for the addition of the perturbation, if necessary.

Spectroscoping/Detecting Part (Spectroscoping Means and Detecting Means)

The measurement system of the present apparatus has a construction of the near-infrared spectroscope. The near-infrared spectroscope generally irradiates a test sample, an object to be measured, with light, of which the reflection light, the transmission light or the transmission reflection light from an object is detected by the detecting part. Further, the wavelength-depending absorbance of the detected light to an incident light is measured.

The examination/diagnosis apparatus for a cancer patient, particularly a hepatic cancer patient, preferably measures the absorbance at two or more wavelengths selected from a plurality of ±5 nm wavelength ranges in respective wavelengths of 625 to 675 nm, 775 to 840 nm, 910 to 950 nm, 970 to 1010 nm, 1020 to 1060 nm, and 1070 to 1090 nm.

Further, the examination/diagnosis apparatus for an SLE patient preferably measures the absorbance at two or more wavelengths selected from a plurality of ±5 nm wavelength ranges in respective wavelengths of 740 to 780 nm, 790 to 840 nm, 845 to 870 nm, 950 to 970 nm, 975 to 1000 nm, 1010 to 1050 nm, and 1060 to 1100 nm.

Furthermore, the examination/diagnosis apparatus for an antiphospholipid antibody syndrome (APLs positive or negative) preferably measures the absorbance at two or more wavelengths selected from a plurality of ±5 nm wavelength ranges in respective wavelengths of 600 to 650 nm, 660 to 690 nm, 780 to 820 nm, 850 to 880 nm, 900 to 920 nm, 925 to 970 nm, and 1000 to 1050 nm.

Spectroscopy system is divided into pre-spectroscopy and post-spectroscopy. The former spectroscopes lights before the irradiation to the object to be measured. The latter detects light from the object to be measured to spectroscope lights. The spectroscope/detector of the present apparatus may adopt any system of pre-spectroscopy and post-spectroscopy.

There are three kinds of detection methods: reflection light detection method, transmission light detection method, and transmission reflection light detection method. In the reflection light detection and the transmission light detection method, the reflection light and the transmission light from the object to be measured are detected respectively by a detector. The transmission reflection light detection method detects the light which incident light refracts and reflects inside the object to emit again outside the object and interfere with the reflection light. The spectroscoping/detecting part of the present apparatus may adopt any system of reflection light detection method, transmission light detection method, and transmission reflection light detection method.

The detector in the spectroscoping/detecting part, for example, may comprise, but is not limited to, a CCD (Charge Coupled Device) which is a semiconductor device, and may adopt other light-receiving devices. The spectroscope can be comprised of a known means.

Data Analyzing Part (Data Analyzing Means)

The wavelength-depending absorbance which is an absorbance spectroscopic data is provided by the spectroscoping/detecting part. Based on the absorbance spectroscopic data, the data analyzing part use the analysis model previously prepared to identify changes in a test sample environment.

In analysis models, a plurality of analysis models such as a quantitative model and a qualitative model are prepared and any of them may be appropriately used depending on whether the data is quantitatively or qualitatively evaluated. Further, an analysis models are preferably prepared based on each amount of substance related to cancer, systemic lupus erythematosus (SLE), or antiphospholipid antibody syndrome, and may be designed to be able to perform any of examinations with a single apparatus.

The data analyzing part can comprise a storage part for storing various data such as a spectroscopic data, a multivariate analysis program and an analysis model, and an arithmetic processing part for processing these data and the program arithmetically. The storage and operation can be achieved, for example, by an IC chip. Therefore, the present apparatus can be easily small-sized to be a handheld one. The analysis model as described above is also written in the storage part such as the IC chip.

Result Displaying Part (Displaying Means)

The result displaying part displays an analysis result obtained by the data analyzing part. Specifically, it displays the concentration value such as the amount of particular biochemical substance given by analysis using an analysis model in the test sample. Alternatively, based on the judgment result given by analysis using the qualitative model, it displays “normal”, “highly likely to be abnormal”, or “abnormal”. The result display is preferably to be a flat display such as liquid crystal when the apparatus is used as a portable one.

The present invention will be described in reference to Examples below, but is not limited by the Examples.

### Example 1

Examination with Near-Infrared Spectrometry

Measurement of Absorption Spectrum

The present Example used a following measurement method to measure the absorption spectrum of each test sample.

A normal person serum and each clinic disease sample (cancer, systemic lupus erythematosus (SLE), or antiphospholipid antibody syndrome) serum were obtained and diluted about 20 times to use as test samples. The three absorbance data respectively obtained by three times-continuous irradiation per test sample are used to prepare a analysis model. An analysis model can be prepared by such a way, and an unknown sample can be measured by spectrometry in the similar way to give absorbance data, which are then analyzed using the analysis models to allow the examination/diagnosis of each disease (cancer, systemic lupus erythematosus (SLE), or antiphospholipid antibody syndrome).

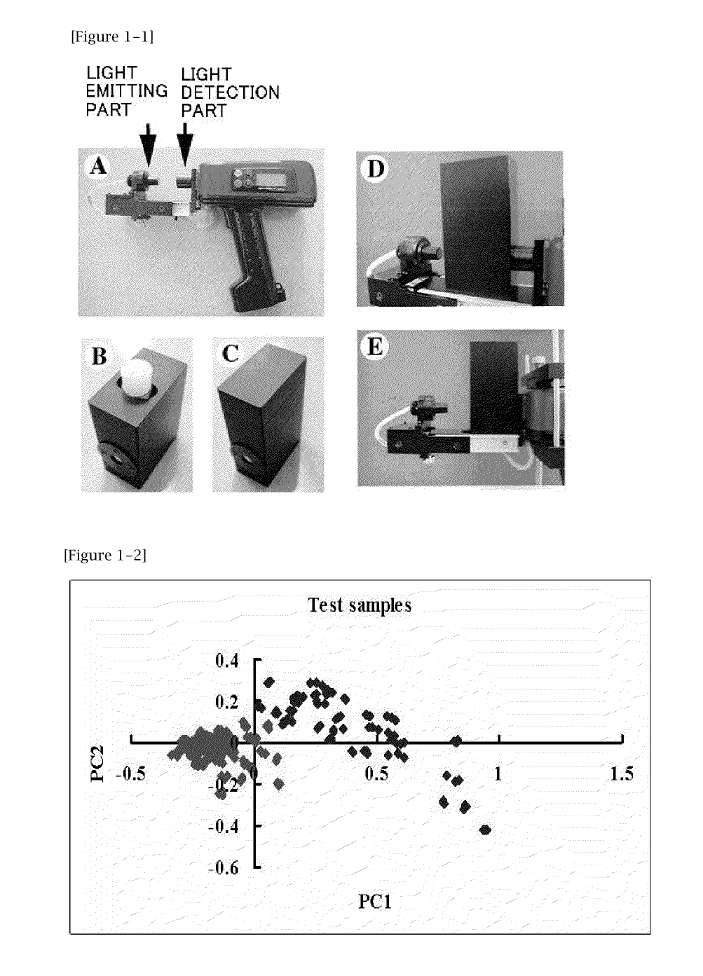

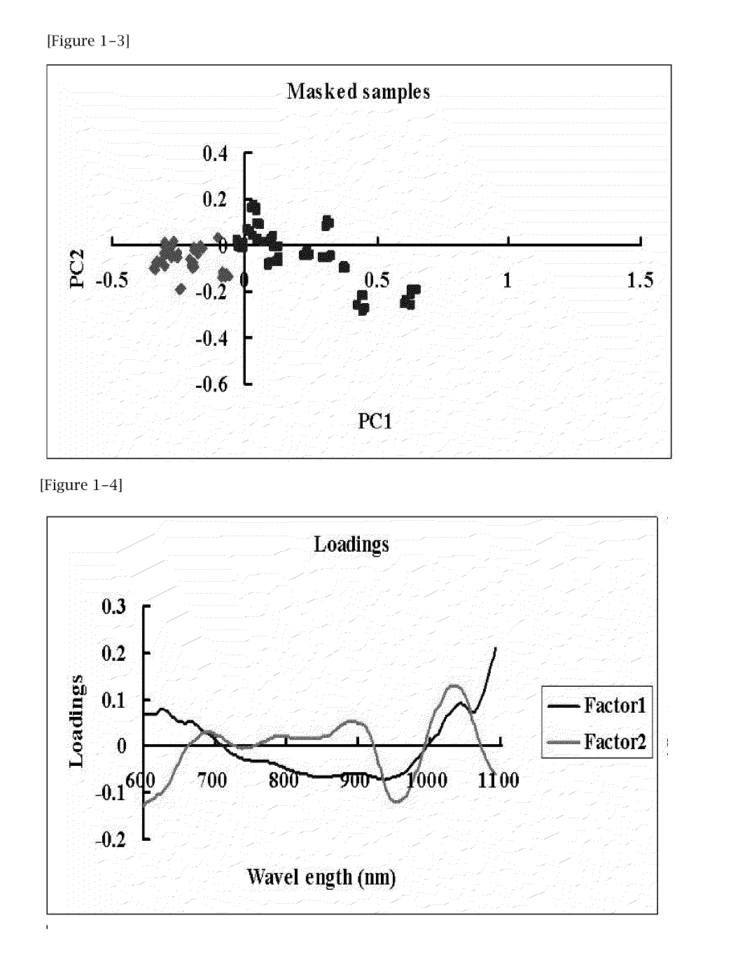

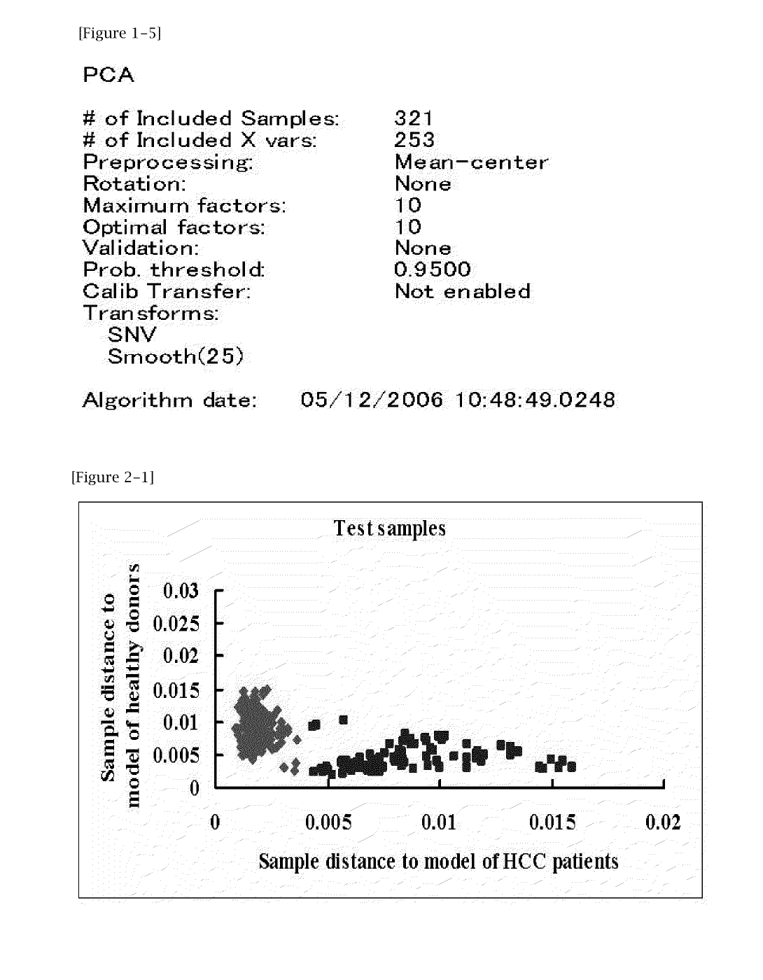

In each of the group, each of the sera as a test sample was measured using near-infrared light. The test sample was diluted about 10 times to put in a polystyrene cuvette, and was measured using the near-infrared spectrometry (FQA-NIRGUN [Japan Fantec Research Institute, Shizuoka, Japan]) while adding the perturbation of repeated irradiation with lights. Specifically, the test sample was irradiated continuously three times with light having a wavelength of 600-1100 nm to detect their respective transmission lights, thereby to measure absorption spectra. The wavelength resolution is 2 nm. As shown in FIG. 1-1, the length of the light path across the test sample was set to a size of a test sample vessel, because the test sample was held between light emitting parts and light detection parts. (see: Akikazu Sakudou, Takanori Kobayashi, Yoshikazu Suganuma, Yukiyoshi Hirase, Hirohiko Kuratsune, Kazuyoshi Ikuta. Special topic; Fatigue/Boredom, the novel diagnostic method of fatigue “the diagnostic method using near-infrared spectroscopy analysis,” Sogo rinsho, vol. 55, p 70-75, 2006)

### Example 2

Analysis of Absorption Spectrum

In the present Example, absorption spectra of a normal person's blood and each clinical disease (cancer, systemic lupus erythematosus (SLE), or antiphospholipid antibody syndrome) patient's blood were measured, and then regarding their differences, a principal component analysis or SIMCA analysis was performed to allow the preparation of a principal component analysis model and a SIMCA model, thereby to analyze the magnitude of the difference at respective wavelength of respective diseases (cancer, systemic lupus erythematosus (SLE), or antiphospholipid antibody syndrome) patient and a normal person at the respective wavelengths and to investigate the analysis data. Regarding antiphospholipid antibody syndrome, antiphospholipid antibody was also estimated/examined positive or negative.

Estimation for an unknown test sample with the model prepared as described above was decided as follows.

Masked sample was prepared apart from Test sample used for the preparation of the model and the Masked sample was used as an unknown test sample to be measured for the estimation. The absorption spectrum of these test samples to be measured for the estimation was substituted to the principal component analysis model and the SIMCA model to examine the efficacy of the model.

There is a method for verifying the efficacy of the model by using the method called validation. The validation is the method which verifies the efficacy of the model by removing the sample and comprises mainly step validation and cross validation. The step validation excludes a group sequencing sample, and on the other hand, the cross validation excludes infrequent sample, and then after preparing a model, whether the samples excluded are justly identified or not, will be verified. Such validation was not carried out this time in this Examples, as the efficacy of the model was investigated using an unknown test sample.

The result will be described below.

FIGS. 1 (2 to 4) shows a Score result of a principal component analysis of hepatic cancer patients (HCC) and normal persons by a measurement using near-infrared spectroscopy. FIG. 1-2 and FIG. 1-4 show a preparation result of the analysis model for the principal component analysis in Test sample (hepatic cancer patient; 76, normal person; 31) by the near-infrared spectroscopy.

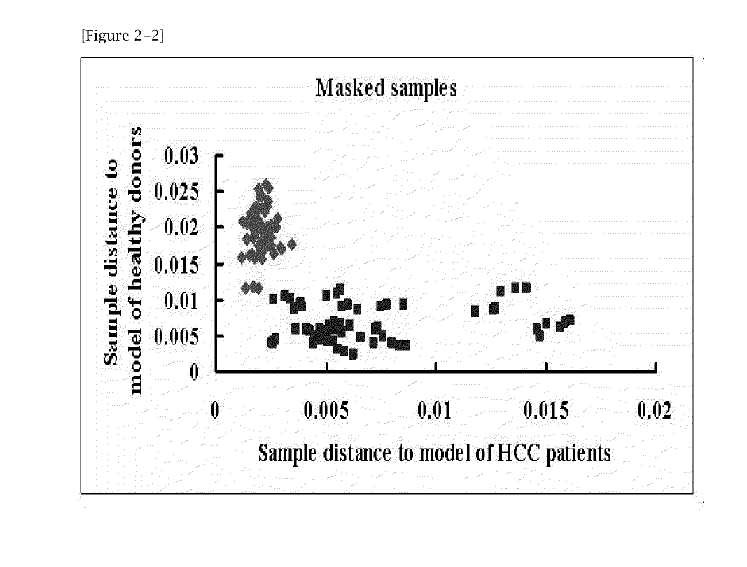

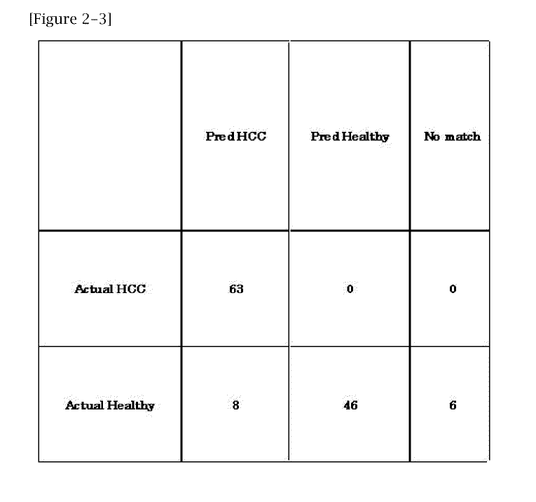

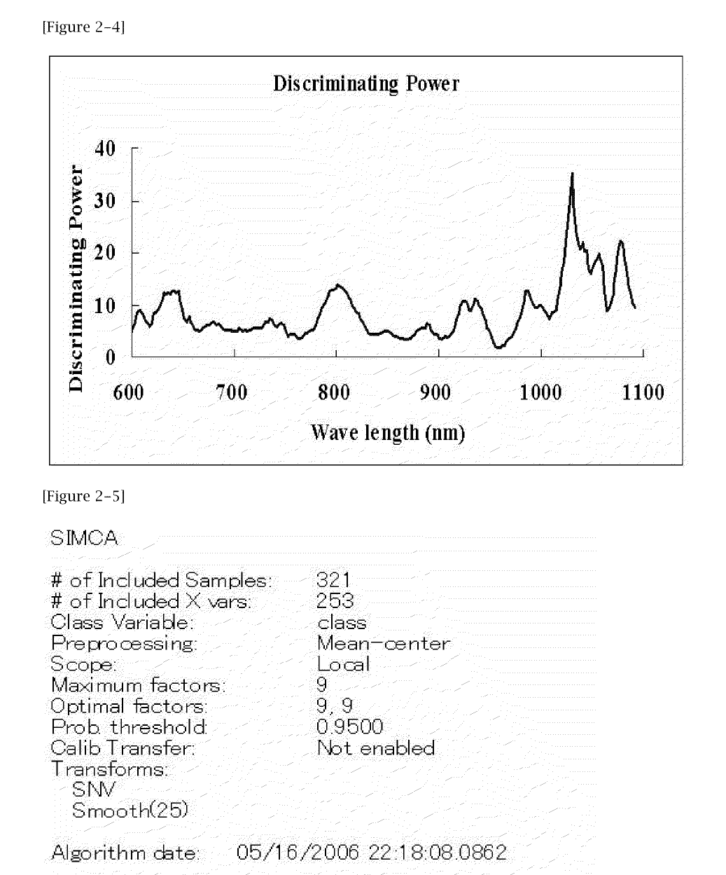

FIG. 1-2, which shows PC2 (Score of a principal component 2) at the vertical axis and PC1 (Score of a principal component 1) at the horizontal axis, is to perform the distributive analysis of spectra of hepatic cancer (HCC) patients and normal person spectra at plotting positions from a PC1 & PC2 of each test sample. As a result, the plottings of the spectra of hepatic cancer (HCC) patients were distributed on the left-side as gray display of FIG. 1-2 and the plottings of the normal person spectra were distributed on the right-side as black display of FIG. 1-2.

FIG. 1-3 shows a result identifying the masked samples (hepatic cancer patient; 21, normal person; 20) using a PCA by the measurement with the near-infrared spectrometry. FIG. 1-3, which shows PC2 (Score of a principal component 2) at the vertical axis and PC1 (Score of a principal component 1) at the horizontal axis, is a result of an distributive analysis of patients with hepatic cancer and normal persons at the plotting positions from the PC1 & PC2 of each test sample. As a result, the plottings of the spectra of hepatic cancer (HCC) patients were distributed on the left-side as gray display of FIG. 1-3 and the plottings of the spectra of normal person were distributed on the right-side as black display of FIG. 1-3.

FIG. 1-4 shows a Loading in each of the wavelengths between the principal component 1 and the principal component 2. Black and gray are the cases of the principal component 1 and the principal component 2, respectively. The principal component 1 seriously uses the absorbance of 630 nm, 800 to 950 nm, and 1050 nm, and the principal component 2 seriously uses the absorbance of 630 nm, 700 nm, 900 nm, 950 nm, and 1050 nm.

FIG. 1-5 shows an analytical condition of the principal component. The algorithms of FIG. 1-5 will be briefly described.

“# of Included Samples” is a sample number (spectrum number) used for the analysis, and a sample number of 321 means that 107 samples were irradiated continuously three times to give three respective absorbance data, which were then used.

“Preprocessing” means a pre-treatment, and “Mean-center” shows that an original point for plotting is shifted to the center of a data set. “Maximum factor” is a Factor (principal component) number to analyze at the maximum, and up to 10 was selected. “Optimal Factors” shows an optimal Factor number for preparing a model which is found out from analysis result.

“Prob. threshold” is a threshold value used to determine whether a subject belongs to a certain class or not. “Calib Transfer” shows whether mathematical adjustment is required to alleviate the difference between apparatuses or not. “Transform” shows a transformation, and “Smooth” shows smoothing.

FIGS. 2 (1 to 5) shows a result of SIMCA analysis of a hepatic cancer (HCC) patient and a normal person by measurement with near-infrared spectroscopy.

FIG. 2-1 shows a preparation result of the principal component analysis model using Test sample (hepatic cancer patient; 76, normal person; 31) by measurement with near-infrared spectroscopy, and the horizontal axis shows a distance of each spectrum (different degree) from the typical spectra of hepatic cancer (HCC) patients defined by the SIMCA model. The vertical axis shows a distance of each spectrum from the typical spectra of normal persons defined by the SIMCA model. The spectra of normal persons are indicated by the black plottings on the right side of FIG. 2-1, and the spectra of hepatic cancer (HCC) patients are indicated by gray plottings on the left side of FIG. 2-1.

The FIG. 2-2 shows the identification result using masked sample (hepatic cancer patient; 21, normal person; 20), and the horizontal axis shows a distance of each spectrum (different degree) from the typical spectra of hepatic cancer (HCC) patients defined by the SIMCA model. The vertical axis shows a distance of each spectrum from the typical spectra of normal persons defined by the SIMCA model. The spectra of normal persons are indicated by the black plottings on the right side of FIG. 2-2, and the spectra of hepatic cancer (HCC) patients are indicated by the gray plottings on the left side of FIG. 2-2.

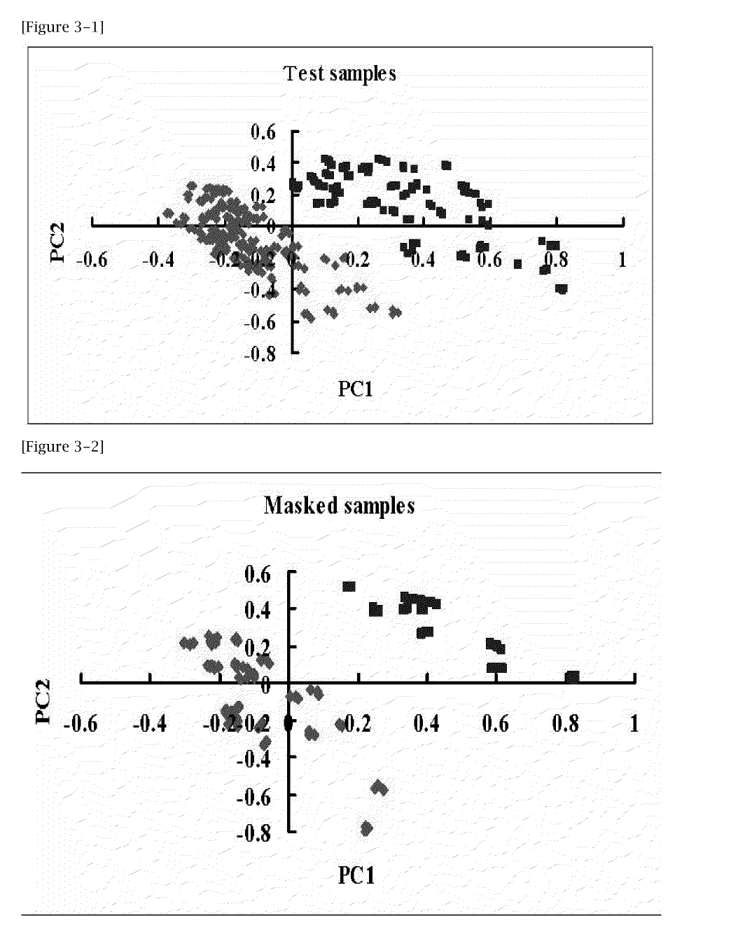

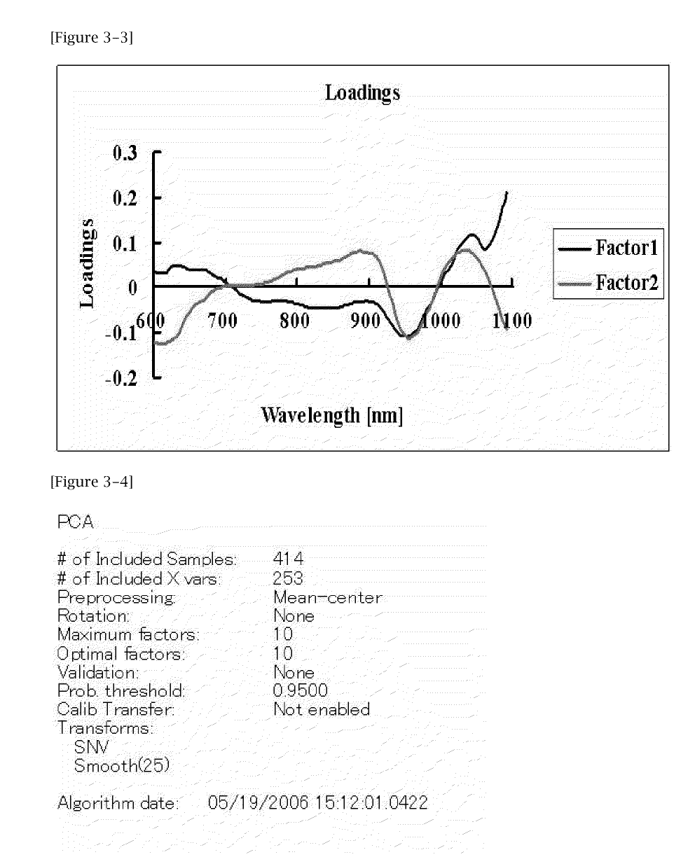

FIG. 2-3 shows a prediction result of cancer from the SIMCA model, and a result of Masked samples: hepatic cancer patient 21.times.3 spectra and normal person 20.times.3 spectra. The vertical axis shows real numbers of spectra of hepatic cancer (HCC) patients and spectra of normal persons, and pred HCC and Pred Healthy of the horizontal axis are the prediction result by the SIMCA model: 63 cases in which an actual spectrum of hepatic cancer (HCC) patients was estimated to be a spectrum of hepatic cancer (HCC) patients by the SIMCA model and the results coincided with each other; 8 cases in which an actual spectrum of normal persons was identified to be a spectrum of hepatic cancer (HCC) patients by the SIMCA model; 0 cases in which an actual spectrum of hepatic cancer (HCC) patients was estimated to be a spectrum of normal persons by the SIMCA model; 46 cases in which predicted an actual spectrum of normal persons to be a spectrum of normal persons by the SIMCA model; the term “NO MATCH” used in the table means the cases where it was not estimated to be neither a spectrum of hepatic cancer (HCC) patients nor a spectrum of normal persons.

FIG. 2-4 shows a wavelength at a horizontal axis and a discriminating power (showing at which wavelengths there are statistically differences in absorption between spectra of hepatic cancer patients and spectra of normal persons) at a vertical axis. Thus, the wavelength, which corresponds to sharp peak having high discriminating power, is considered to be one of effective wavelengths for distinguishing between normal persons and hepatic cancer (HCC) patients. Therefore, through the identification by focusing attention on the wavelength described in FIG. 2-4, obtained by the SIMCA analysis, it is possible to carry out simple, prompt, and accurate diagnosis as to whether the sample belongs to hepatic cancer (HCC) patients or not.

With the result of FIG. 2-4, the present invention could perform the examination/identification/diagnosis of patients with cancer, particularly hepatic cancer (HCC), through the analysis using the absorption spectroscopic data at two or more wavelengths, which are selected from a plurality of ±5 nm wavelength ranges of wavelengths selected from the group consisting of 625 to 675 nm, 775 to 840 nm, 910 to 950 nm, 970 to 1010 nm, 1020 to 1060 nm, and 1070 to 1090 nm.

Further, FIG. 2-5 shows conditions of SIMCA. Algorithms shown in FIG. 2-5 will be briefly explained as below.

“# of Included Samples” is a sample number (spectrum number) used for the analysis, and a sample number of 321 means that 107 samples were irradiated continuously three tomes to give three respective absorbance data, which were then used.

“Preprocessing” means a pre-treatment, and “Mean-center” shows that an original point for plotting is shifted to the center of a data set. “Scope” includes a Global one and a Local one, and the Local one was selected. “Maximum factors” is a Factor (principal component) number to analyze at the maximum, and up to 9 was selected. “Optimal Factors” is an optimal Factor number for preparing a model which is found out from analysis result. “Prob. threshold” is a threshold value used to determine whether a subject belongs to a certain class or not. “Calib transfer” shows whether mathematical adjustment is required to alleviate the difference between apparatuses or not. “Transform” shows a transformation, and “Smooth” shows smoothing.

FIGS. 3 (1 to 4) showed a Score result of the principal component analysis between systemic lupus erythematosus (SLE) and normal persons. FIG. 3-1 and FIG. 3-3 show a preparation result of the analysis model for the principal component analysis using Test samples (patient with SLE; 97, normal person; 41). FIG. 3-2 shows an identification result using masked samples (SLE; 25, normal person; 10) by a near-infrared spectrum.

FIG. 3-1, which shows PC2 (Score of a principal component 2) at the vertical axis and PC1 (Score of a principal component 1) at the horizontal axis, is a result of a distributive analysis of SLE patients and normal persons at plotting positions from the PC1 & PC2 of each test sample. As a result, the plottings of the spectra of patients with SLE were distributed on the left-side as gray display of FIG. 3-1 and the plottings of the spectra of normal persons were distributed on the right-side as black display of FIG. 3-1.

FIG. 3-2 shows an identification result in masked samples using the principal component analysis with the near-infrared spectrum. FIG. 3-2, which shows PC2 (Score of a principal component 2) at the vertical axis and PC1 (Score of a principal component 1) at the horizontal axis, is a result of a distributive analysis of SLE patients and normal persons at plotting positions from the PC1 & PC2 of each test sample. As a result, the plottings of spectra of patients with SLE were distributed on the left-side as gray display of FIG. 3-2 and the plottings of the spectra of normal persons were distributed on the right-side as black display of FIG. 3-2.

FIG. 3-3 shows a Loading in each of the wavelengths between the principal component 1 and the principal component 2. Black and gray are cases of the principal component 1 and the principal component 2, respectively. The principal component 1 seriously uses 650 nm, 800 to 900 nm, 950 nm and 1050 nm, and the principal component 2 is seriously uses 620 nm, 900 nm, 950 nm, and 1050 nm.

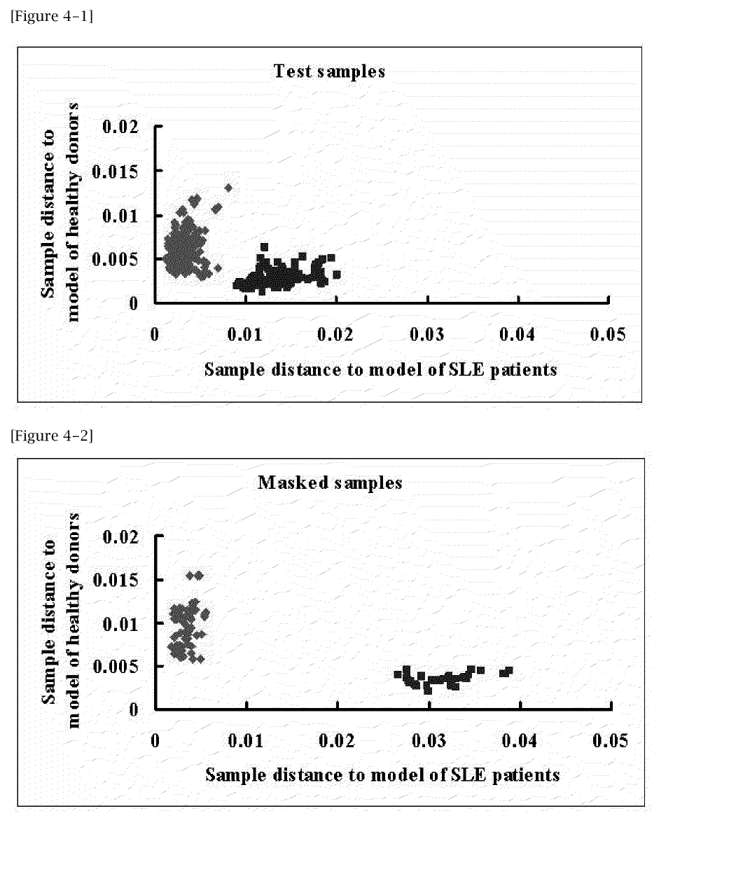

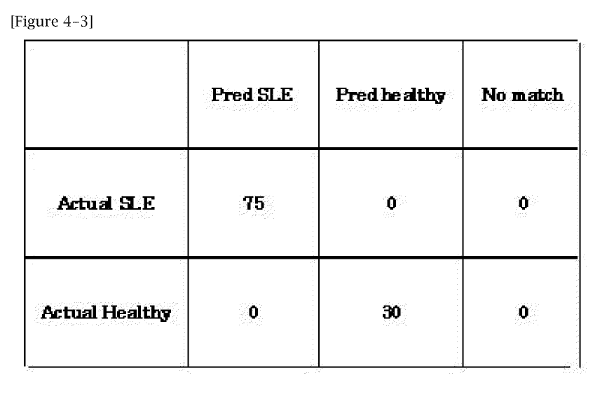

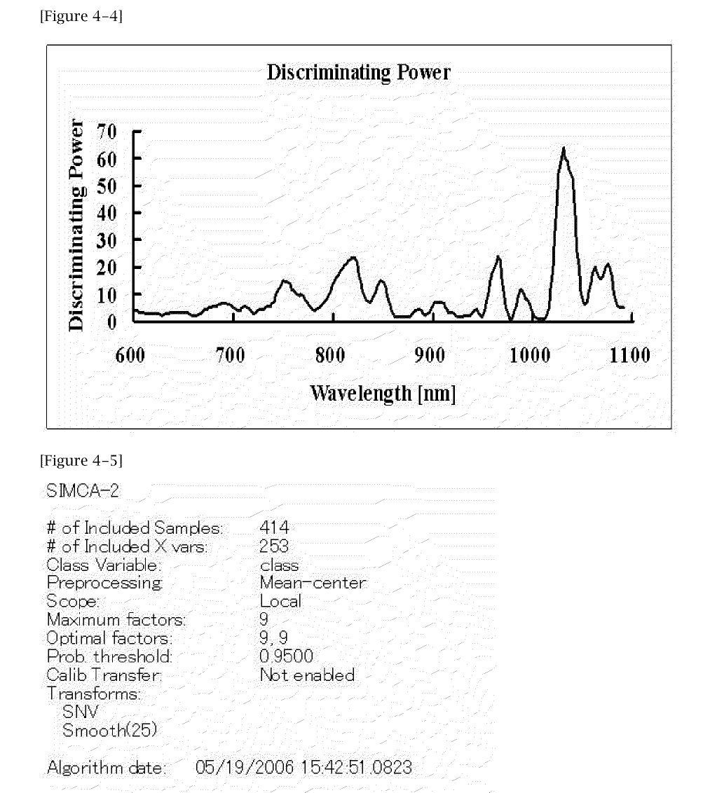

Further, FIG. 3-4 shows analysis conditions of the principal component (see, the brief description of algorithm shown in FIG. 1).

FIGS. 4 (1 to 5) shows a result of SIMCA of patients with SLE and normal persons by measurement using near-infrared spectroscopy. FIG. 4-1 and FIG. 4-4 showed a preparation result of the SIMCA model using Test sample (patient with SLE; 76, normal person; 31) by the near-infrared spectrum. The horizontal axis of FIG. 4-1 shows a distance of each spectrum (different degree) from the typical spectra of SLE patients defined by the SIMCA model. The vertical axis shows a distance of each spectrum from the typical spectra of normal persons defined by the SIMCA model.

The spectra of normal persons are indicated by the black plottings on the right side of FIG. 4-1, and the spectra of patients with SLE are indicated by the gray plottings on the left side of FIG. 4-1.

The FIG. 4-2 shows an identification result using masked sample (patient with SLE; 25, normal person; 10), and the horizontal axis shows a distance of each spectrum (different degree) from the typical spectra of SLE patients defined by the SIMCA model. The vertical axis shows a distance of each spectrum from the typical spectra of normal persons defined by the SIMCA model. The spectra of normal persons were black plottings on the right side of FIG. 4-2, and the spectra of SLE patients were gray plottings on the left side of FIG. 4-2.

FIG. 4-3 shows a prediction result of SLE from the SIMCA model, and a result of Masked samples: patient with SLE 25.times.3 spectra and normal person 10.times.3 spectra. The vertical axis shows real numbers of both patients with SLE and normal persons, and Pred SLE and Pred Healthy of the horizontal axis are the prediction by the SIMCA model: 75 cases in which an actual spectrum of SLE patients was estimated to be a spectrum of SLE patients by the SIMCA model and the results coincided with each other; 0 cases in which the actual spectrum of normal persons was identified to be a spectrum of SLE patients by the SIMCA model; 0 cases in which the actual spectra of SLE patients was estimated to be a spectrum of normal person by the SIMCA model; 30 cases in which the actual spectrum of normal persons was estimated to be a spectrum of normal persons by the SIMCA model; the term “NO MATCH” used in the Table means the case where it was not estimated to be neither a spectrum of SLE patients nor a spectrum of normal persons.

FIG. 4-4 shows a wavelength at a horizontal axis and a discriminating power (showing at which wavelengths there are statistically differences in absorption between spectra of SLE patients and spectra of normal persons) at a vertical axis. Namely, the wavelength, which corresponds to sharp peak having high discriminating power, is considered to be one of effective wavelengths for distinguishing between normal persons and SLE patients. Therefore, through the identification by focusing attention on the wavelength described in FIG. 4-4, obtained by the SIMCA analysis, it is possible to carry out simple, prompt, and accurate diagnosis as to whether the sample belongs to SLE patients or not.

With the result of FIG. 4-4, the present invention could perform the examination/identification/diagnosis of patients with SLE through the analysis using the spectroscopic absorption data at two or more wavelengths, which are selected from a plurality of ±5 nm wavelength ranges of wavelengths selected from the group consisting of 740 to 780 nm, 790 to 840 nm, 845 to 870 nm, 950 to 970 nm, 975 to 1000 nm, 1010 to 1050 nm, and 1060 to 1100 nm.

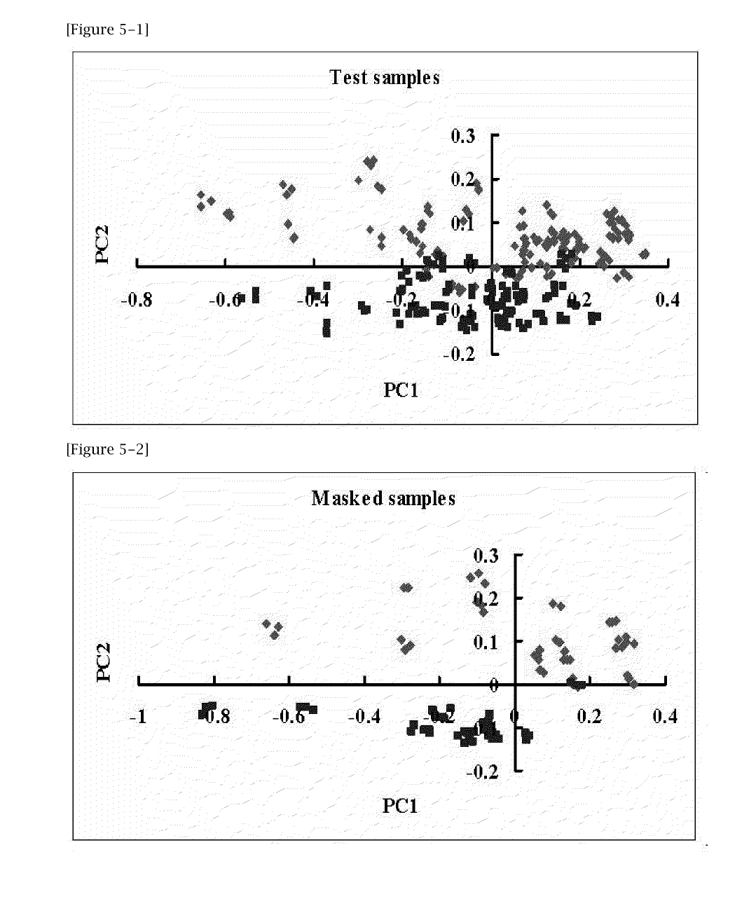

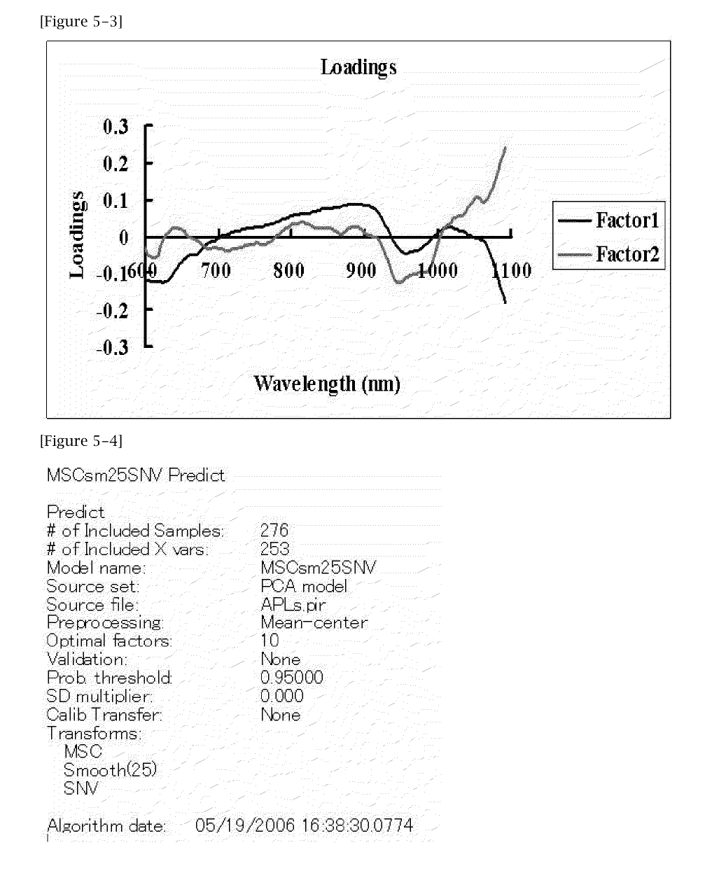

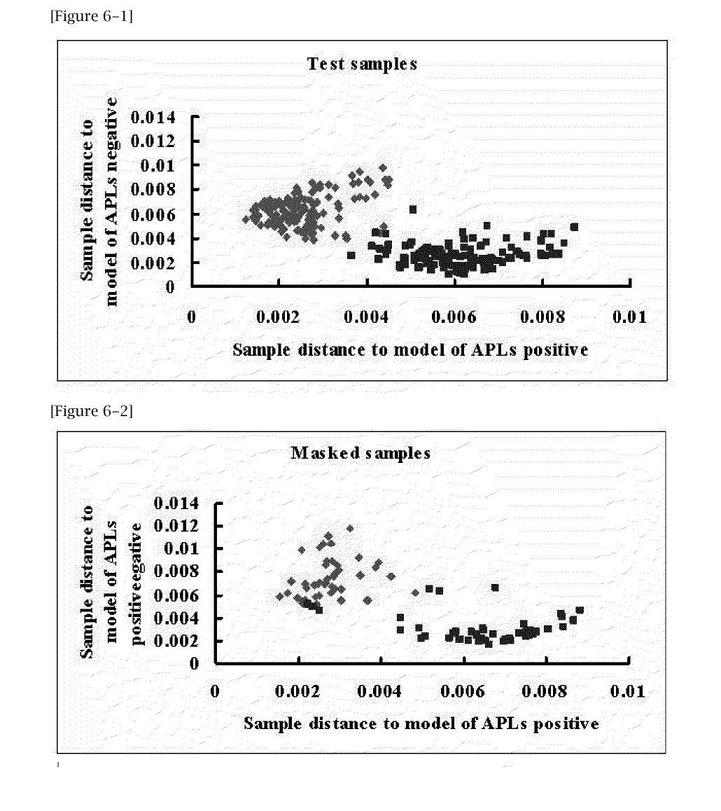

Further, FIG. 4-5 shows conditions of SIMCA (see, the brief description of algorithm shown in FIG. 2).

FIGS. 5 (1 to 4) showed a Score result of the principal component analysis between the test sample of antiphospholipid antibody (APLs) positive in systemic lupus erythematosus (SLE) and the test sample of APLs negative in SLE. FIG. 5-1 and FIG. 5-3 show a preparation result of the analysis model for a principal component analysis using Test sample (APLs (+); 51, APLs (−); 41). FIG. 5-2 shows an identification result using masked samples (APLs (+); 15, APLs (−); 15) by the near-infrared spectrum.

FIG. 5-1, which shows PC2 (Score of a principal component 2) at the vertical axis and PC1 (Score of a principal component 1) at the horizontal axis, is a result of a distributive analysis of the spectra of APLs positive patients and the spectra of APLs negative patients at plotting positions from the PC1 & PC2 of each test sample. As a result, the plottings of the spectra of APLs positive patients were distributed on the upper side as gray display of FIG. 5-1 and the plottings of the spectra of APLs negative patients were distributed on the downside as black display of FIG. 5-1.

FIG. 5-2 shows an identification result in masked samples using the principal component analysis Score with the near-infrared spectrum. FIG. 5-2, which shows PC2 (Score of a principal component 2) at the vertical axis and PC1 (Score of a principal component 1) at the horizontal axis, is a result of a distributive analysis of the spectra of APLs positive patients and the spectra of APLs negative patients at plotting positions from the PC1 & PC2 of each test sample. As a result, the plottings of the spectra of APLs positive patients were distributed on the upper side as gray display of FIG. 5-2 and the plottings of the spectra of APLs negative patients were distributed on the downside as black display of FIG. 5-2.

FIG. 5-3 shows a Loading result in each of the wavelengths between the principal component 1 and the principal component 2. Black and gray are cases of the principal component 1 and the principal component 2, respectively. The principal component 1 seriously uses 620 nm, 905 nm, 960 nm and 1020 nm, and the principal component 2 seriously uses 640 nm, 810 nm, 940 nm, 1020 nm, and 1060 nm.

Further, FIG. 5-4 shows analysis conditions of the principal component (see, the brief description of algorithm shown in FIG. 1).

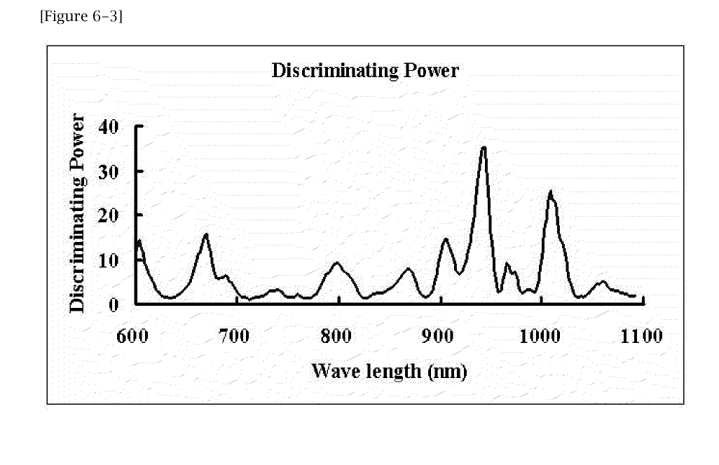

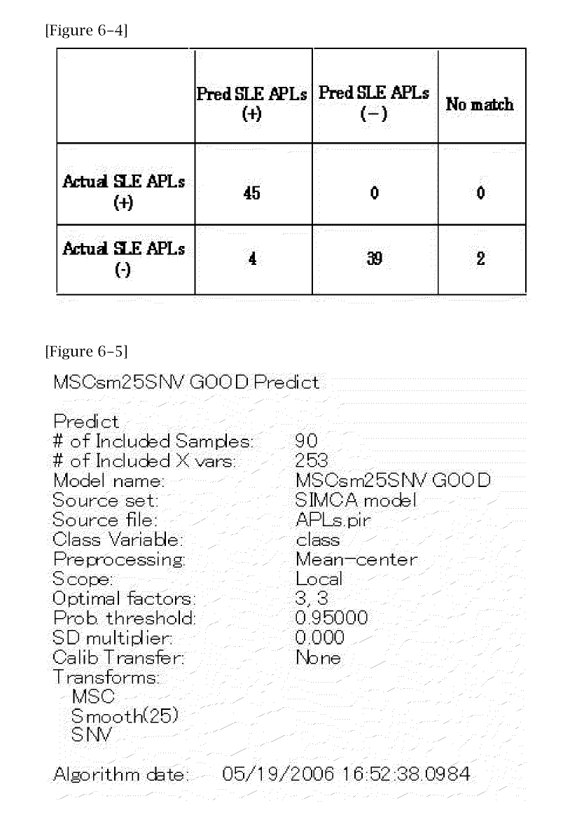

FIGS. 6 (1 to 5) showed a SIMCA analysis result between the test sample of antiphospholipid antibody (APLs) positive in systemic lupus erythematosus (SLE) and the test sample of APLs negative in SLE. FIG. 6-1 and FIG. 6-3 show a preparation result of the SIMCA model using Test sample (APLs positive patient; 51, APLs negative patient; 41) by the near-infrared spectrum.

The horizontal axis of FIG. 6-1 shows a distance of each spectrum (different degree) from the typical spectra of APLs positive patients defined by the SIMCA model. The vertical axis shows a distance of each spectrum from the typical spectra of APLs negative patients defined by the SIMCA model. The spectra of APLs negative patients are indicated by the black plottings on the right downside of FIG. 6-1, and the spectra of APLs positive patients are indicated by the gray plottings on the left upper-side of FIG. 6-1.

The FIG. 6-2 shows an identification result using masked sample (APLs positive patient; 15, APLs negative patient; 15), and the horizontal axis shows a distance of each spectrum (different degree) from the typical spectra of APLs positive patients defined by the SIMCA model. The vertical axis shows a distance of each spectrum from the typical spectra of APLs negative patients defined by the SIMCA model. The spectra of APLs negative patients are indicated by the black plottings on the right downside of FIG. 6-2, and the spectra of APLs positive patients are indicated by the gray plottings on the left upper-side of FIG. 6-2.

FIG. 6-3 shows a wavelength at a horizontal axis and a discriminating power (showing at which wavelengths there are statistically differences in absorption between spectra of APLs positive patients and spectra of APLs negative patients) at the vertical axis. Thus, the wavelength, which corresponds to a sharp peak having a high discriminating power, is considered to be one of effective wavelengths for distinguishing between APLs positive patients and APLs negative patients. Therefore, the wavelength according to FIG. 6-3, obtained by the SIMCA analysis, is used to identify, allowing simple, prompt, and accurate diagnosis as to whether the sample belongs to APLs positive patients or APLs negative patients. With the result of FIG. 6-3, the present invention could perform the examination/identification/diagnosis of antiphospholipid antibody syndrome (APLs; positive or negative) through the analysis using the absorption spectroscopic data at two or more wavelengths, which are selected from a plurality of ±5 nm wavelength ranges of wavelengths selected from the group consisting of 600 to 650 nm, 660 to 690 nm, 780 to 820 nm, 850 to 880 nm, 900 to 920 nm, 925 to 970 nm, and 1000 to 1050 nm.

FIG. 6-4 shows a prediction result of APLs positive patients from SIMCA model, and are the results in Masked sample: APLs positive patients 25.times.3 spectra and APLs negative patients 10.times.3 spectra. The vertical axis shows real numbers of APLs positive patients or APLs negative patients, and Pred APLs (+) and Pred APLs (−) of the horizontal axis are prediction results from the SIMCA model: 45 cases in which an actual spectrum of APLs positive patients was estimated to be a spectrum of APLs positive patients by the SIMCA model and the results coincided with each other; 4 cases in which the actual spectrum of APLs negative patients was identified to be a spectrum of APLs positive patients by the SIMCA model; 0 cases in which the actual spectrum of APLs positive patients was estimated to be a spectrum of APLs negative patients by the SIMCA model; 39 cases in which the actual spectrum of APLs negative patients was estimated to be a spectrum of APLs negative patients by the SIMCA model; the term “NO MATCH” used in the Table means the cases where it was not estimated to be neither a spectrum of APLs positive patients nor a spectrum of APLs negative patients.

Further, FIG. 6-5 shows conditions of SIMCA (see, the brief description of algorithm shown in FIG. 2).

### INDUSTRIAL APPLICABILITY

As described above, the present invention can examine/identify simply, promptly, and accurately cancer, systemic lupus erythematosus (SLE), or antiphospholipid antibody syndrome of blood and blood-derived component, wherein blood and blood-derived component are irradiated with lights having a wavelength of 400 nm to 2500 nm or a part of the range, of which the reflection light, the transmission light, or the transmission reflection light is then detected to give spectroscopic absorbance data, and afterward a previously prepared analysis model is used to analyze the absorbance over the whole wavelengths or at a specific wavelength for the measurement, and thus can be widely used for clinical examinations and the like.

1. A method for identification of a clinical disease selected from following items, comprising the steps of:
irradiating collected blood, blood-derived component, urine, sweat, nail, skin, or hair with light having a wavelength of 400 to 2500 nm or a part of the range, of which
a reflection light, a transmission light, or a transmission reflection light is then detected to give spectroscopic absorbance data, and
analyzing an absorbance over the whole wavelengths or at a specific wavelength for a measurement through the use of a previously prepared analysis model of
1) Cancer,
2) Systemic lupus erythematosus (SLE), or
3) Antiphospholipid antibody syndrome.

2. The method for identification according to claim 1, comprising the steps of:
irradiating the blood, blood-derived component, urine, sweat, nail, skin, or hair collected from normal persons and patients with clinical disease with light having the wavelength of 400 to 2500 nm or a part of the range, of which
the reflection light, the transmission light, or the transmission reflection light is then detected to give spectroscopic absorbance data, and
analyzing the difference wavelength after assaying the difference in absorbance between normal persons and patients with clinical disease.

3. The method for identification according to claim 2, wherein an analysis method of the difference wavelength adopts a principal component analysis or a SIMCA method.

4. The method for identification according to claim 1, wherein a perturbation is given to the collected blood, blood-derived component, urine, sweat, nail, skin, or hair.

5. The method for identification according to claim 1, wherein the absorption spectrum detected is the transmission light.

6. The method for identification according to claim 1, wherein the absorption spectroscopic data at two or more wavelengths, which are selected from a plurality of ±5 nm wavelength ranges of each wavelength selected from the group consisting of 625-675 nm, 775-840 nm, 910-950 nm, 970-1010 nm, 1020-1060 nm, and 1070-1090 nm, are used for the identification of a clinical disease of cancer.

7. The method for identification according to claim 1, wherein the absorption spectroscopic data at two or more wavelengths, which are selected from a plurality of ±5 nm wavelength ranges of each wavelength selected from the group consisting of 740-780 nm, 790-840 nm, 845-870 nm, 950-970 nm, 975-1000 nm, 1010-1050 nm, and 1060-1100 nm, are used for the identification of the clinical disease of systemic lupus erythematosus (SLE).

8. The method for identification according to claim 1, wherein the absorption spectroscopic data at two or more wavelengths, which are selected from a plurality of ±5 nm wavelength ranges of each wavelength selected from the group consisting of 600-650 nm, 660-690 nm, 780-820 nm, 850-880 nm, 900-920 nm, 925-970 nm, and 1000-1050 nm, are used in the identification of the clinical disease of antiphospholipid antibody syndrome.

9. A method for diagnosis of the clinical disease selected from following items, wherein a finger or an ear of a patient with clinical disease is irradiated with light having a wavelength of 400 to 2500 nm or a part of the range, of which the reflection light, the transmission light, or the transmission reflection light is then detected to give spectroscopic absorbance data, and afterward the previously prepared analysis model is used to analyze the absorbance at the whole or specific wavelength used for the measurement of
1) Cancer,
2) Systemic lupus erythematosus (SLE), or
3) Antiphospholipid antibody syndrome.

10. The method for diagnosis according to claim 9, wherein the finger or ear of a normal person and a patient with clinical disease is irradiated with light having the wavelength of 400 to 2500 nm or a part of the range, of which the reflection light, the transmission light, or the transmission reflection light is then detected to give spectroscopic absorbance data, and afterward the analysis model assays the difference of the absorbance between the normal person and the patient with clinical disease to analyze the difference wavelength.

11. An examination/diagnosis apparatus for a clinical disease selected from following items, comprising:
an irradiating means for irradiating blood, blood-derived component, urine, sweat, nail, skin, or hair with the light having a wavelength of 400 to 2500 nm or a part of the range;
a spectroscoping means for spectroscoping before or after irradiation and
a detecting means for detecting the reflection light, the transmission light, or the transmission reflection light of the light irradiated on said blood, blood-derived component, urine, sweat, nail, skin, or hair; and
a data analyzing means for using a previously formed analysis model to analyze the absorbance(s) at the whole or specific wavelength used for the measurement in the absorbance spectroscopic data obtained by the detection, thereby to analyze qualitatively and quantitatively about the blood, blood-derived component, urine, sweat, nail, skin, or hair of
1) Cancer,
2) Systemic lupus erythematosus (SLE), or
3) Antiphospholipid antibody syndrome.

12. The apparatus according to claim 11, wherein the blood, blood-derived component, urine, sweat, nail, skin, or hair of normal persons and patients with clinical disease is irradiated with lights having the wavelength of 400 to 2500 nm or a part of the range, of which the reflection light, the transmission light, or the transmission reflection light is then detected to give spectroscopic absorbance data, and afterward the analysis model is prepared by assaying the difference of the absorbance between the normal person and the patient with clinical disease to analyze the difference wavelength.

13. The apparatus according to claim 12, wherein the analysis method of the difference wavelength adopts the principal component analysis or the SIMCA method.

14. The apparatus according to claim 11, wherein the absorption spectrum detected is the transmission light.

15. The apparatus according to claim 11, wherein the absorption spectroscopic data at two or more wavelengths, which are selected from a plurality of ±5 nm wavelength ranges of each wavelength selected from the group consisting of 625-675 nm, 775-840 nm, 910-950 nm, 970-1010 nm, 1020-1060 nm, and 1070-1090 nm, are used for the clinical disease of cancer.

16. The apparatus according to claim 11, wherein the absorption spectroscopic data at two or more wavelengths, which are selected from a plurality of ±5 nm wavelength ranges of each wavelength selected from the group consisting of 740-780 nm, 790-840 nm, 845-870 nm, 950-970 nm, 975-1000 nm, 1010-1050 nm, and 1060-1100 nm, are used for the clinical disease of systemic lupus erythematosus (SLE).

17. The apparatus according to claim 11, wherein the absorption spectroscopic data at two or more wavelengths, which are selected from a plurality of ±5 nm wavelength ranges of each wavelength selected from the group consisting of 600-650 nm, 660-690 nm, 780-820 nm, 850-880 nm, 900-920 nm, 925-970 nm, and 1000-1050 nm, are used for the clinical disease of antiphospholipid antibody syndrome.
